(19) United States
(12) Patent Application Publication
(10) Pub. No.: US 2026/0004882 A1
(43) Pub. Date: Jan. 1, 2026

(54) AUTOMATIC MASS SPECTROMETRY PEAK SORTING METHOD FOR PROTEIN QUANTIFICATION

(71) Applicant: BERTIS INC, Gyeonggi-do (KR)

(72) Inventors: Dongyoung NOH, Seoul (KR); Seungman HAN, Seoul (KR); Jungkap PARK, Seoul (KR); Sangtae KIM, Gyeonggi-do (KR)

(21) Appl. No.: 18/872,730

(22) PCT Filed: Jun. 5, 2023
(86) PCT No.: PCT/KR2023/007689

§ 371 Date: Dec. 6, 2024

(30) Foreign Application Priority Data
Jun. 7, 2022 (KR) 10-2022-0068726

(51) Int. Cl. G16B 40/10; G01N 30/02; G01N 30/72; G01N 30/86

(52) U.S. Cl. CPC G16B 40/10; G01N 30/7233; G01N 30/8679; G01N 30/8693; G01N 2030/027

(57) ABSTRACT

The present invention relates to an automated peak sorting system having an exceptionally fast processing speed while being as accurate as humans and experts with respect to conventional targeted proteomic peak picking, which requires manual intervention from researchers and wastes a lot of time and resources. A learning model or a computer program capable of executing same, of the present invention, can be useful for rapidly and accurately sorting out a peak optimized for quantification of a plurality of target peptides input as desired by a user through a GUI of the program.

### CROSS-REFERENCE TO RELATED APPLICATIONS

This application is a U.S. National Stage entry of International Patent Application no. PCT/KR2023/007689, filed Jun. 5, 2023, which claims the benefit of priority of Korean Patent Application no. 10-2022-0068726, filed Jun. 7, 2022.

### BACKGROUND OF THE DISCLOSURE

### Technical Field

The present invention relates to a model of interpreting targeted proteomics data based on deep learning algorithms for object detection designed to significantly reduce the time and resource burden of manual peak selection to human expert-level accuracy.

### Background Art

Deep neural network technology has contributed to breakthroughs in discovery proteomics, but its adoption has been slower in targeted proteomics, which focuses on detecting specific target proteins. Meanwhile, in clinical proteomics laboratories, targeted proteomics techniques based on multiplexed reaction monitoring (MRM) or parallel reaction monitoring (PRM) experiments are widely used due to their high sensitivity and reproducibility.

Peak picking is an initial key step in mass spectrometry-based proteomics and a critical step in data analysis. Peak selection, which includes smoothing, baseline correction, and peak alignment, is the preprocessing of data to enable statistical analysis and biological interpretation. Specifically, preprocessing of mass spectrometry data refers to the reduction of large amounts of raw spectral data (typically >30K data points) into a set of statistically valid peaks. Since mass spectrometry data is inevitably noisy due to factors such as the presence of various natural compounds in the sample, such as interference from substrate materials, contamination of the sample or electrical noise that varies depending on the analytical setup, peak selection is essential for accurate analysis.

However, to interpret MRM or PRM data, researchers must manually select peaks, identify interferences, and spend significant time adjusting the area of peaks. This burden of manual inspection is a major factor limiting the reproducibility and scalability of targeted proteomics for clinical applications.

Several methods have been developed to address these issues, including the development of peak selection algorithms and quality control methods, but these methods still require a high degree of manual intervention, making manual inspection a challenge when applied to large-scale proteomics data.

In order to develop a method that can automatically perform data interpretation in targeted proteomics without manual human intervention, the inventors conducted a study to create a deep training model using deep neural network technology and confirmed that the developed model can select peaks with high accuracy while dramatically reducing the time and resources required for data interpretation.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

The inventors have made dedicated effort to develop a method to eliminate the need for human expert intervention in the manual selection of chromatographic peaks, which has been a barrier to rapid and efficient testing in targeted proteomics, and to develop a method to select peaks optimized for rapid quantitation of target peptides with high accuracy.

As a result, the present invention was completed by discovering that a system that includes pre-processing and post-processing of data along with a deep training model with a unique neural network structure developed by the present inventor can select peaks optimized for quantification of target peptides as accurately as human experts when interpreting transition chromatography, while dramatically increasing the processing speed by solving a task that takes over 600 hours for human experts to process in 232 seconds.

Therefore, an object of the present invention is to provide a system for selecting peaks optimized for quantification of target peptides.

Another object of the present invention is to provide a computer program stored on a computer-readable recording medium capable of executing the peak screening system of the present invention to screen peaks optimized for quantification of a target peptide.

Other objects and advantages of the present invention will be more apparent from the following detailed description, the appended claims, and the accompanying drawings.

### Technical Solution

According to one aspect of the present invention, the present invention provides a system for peak selection for quantification of a target peptide in liquid chromatography mass spectrometry (LC-MS) comprising

A preprocessing part that processes the input data for training;

A training part comprising a convolutional neural network (CNN) training model that learns to detect a boundary of a peak optimized for quantification of a target peptide using training data processed in the preprocessing part as input;

A post-processing part that processes the output of the training part; and

A determining part for selecting a peak for quantification of a target peptide using the output of the above post-processing part.

In the present invention, the “liquid chromatography-mass spectrometry”, also referred to as liquid chromatography-mass spectrometry (LC-MS), refers to an analytical chemistry technique that combines the physical separation capabilities of liquid chromatography (or HPLC) with the mass spectrometric capabilities of mass spectrometry (MS). Chromatography and mass spectrometry are widely used in chemical analysis because their combined capabilities synergistically enhance each other. Liquid chromatography separates mixtures containing multiple components, while mass spectrometry provides spectral information to identify each separated component.

In the present invention, the “peak selection” refers to the process of selecting peaks optimized for the quantitation of a target peptide from among the visually identifiable peaks in the data resulting from LC-MS.

In the present invention, the “convolutional neural network”, also known as a CNN, is a type of artificial neural network (ANN) that uses convolutional operations to retain spatial information in an image. Convolutional neural networks are most commonly used for visual image analysis.

In the present invention, the “target peptide” refers to a peptide that is intended to be detected and/or quantified by a mass spectrometry method used in the present invention, and the “target peptide” may be a plurality of peptides.

According to a specific embodiment of the present invention, the mass spectrometry of the present invention is performed by a method selected from the group consisting of Multiple Reaction Monitoring (MRM), Parallel Reaction Monitoring (PRM), Data-Dependent Acquisition (DDA), and Data-Independent Acquisition (DIA).

In the present invention, the “MRM” means multiple reaction monitoring and refers to an analytical technique that can selectively separate, detect, and quantify specific analytes and monitor changes in their concentrations. MRM is a method that can quantitatively and accurately measure multiple substances such as trace biomarkers present in a biological sample, and selectively delivers the parent ion among the ion fragments generated by the ionization source to the collision tube using the first mass filter (Q1). The mother ions reaching the collision tube then collide with the collision gas inside and split to produce daughter ions, which are sent to the second mass filter Q2, where only the characteristic ions are passed to the detector. In this way, it is a highly selective and sensitive analytical method that can detect only the information of the target component. MRM is used for the quantitative analysis of small molecules and is used to diagnose certain genetic diseases. The MRM method is easy to measure multiple peptides simultaneously and has the advantage of being able to determine the relative concentration differences of candidate protein diagnostic markers between normal people and cancer patients without the need for antibodies. In addition, MRM methods are being adopted for the analysis of complex proteins and peptides in blood, especially in proteome analysis using mass spectrometry, due to their excellent sensitivity and selectivity (Anderson L. et al., Mol CellProteomics, 5:375-88, 2006; DeSouza, L. V. et al., Anal. Chem., 81:3462-70, 2009).

In the present invention, the “PRM” refers to parallel reaction monitoring, which is a parallel application of MRM in which all daughter ions generated from one selected parent ion are analyzed simultaneously, as opposed to MRM, which analyzes one parent/daughter ion pair at a time.

In the present invention, the “DIA” refers to Data-Independent Acquisition, which is a method for analyzing all ions in a selected range of m/z values without selecting specific parent ions.

In the present invention, the “DDA” refers to Data-Dependent Acquisition, which, unlike DIA, is a method for selecting a specific parent ion and analyzing only the daughter ions for the selected parent ion.

According to a specific embodiment of the present invention, the data for training is a result of a liquid chromatography mass spectrometry with the predetermined peaks for quantification.

As used herein, “predetermined” refers to the process of manually pre-selecting the best peaks from the MRM data to be used as input for training the training model.

According to a specific implementation of the present invention, the result of a mass spectrometry comprise a transition value of the light peptide and a transition value of the heavy peptide for the target peptide to be quantified.

In the present invention, the “Transition” means a pair of m/z values of a parent ion and a corresponding m/z value of a daughter ion.

In the present invention, the “heavy peptide” refers to a synthetic peptide labeled with an isotope, which may also be referred to as a synthetic isotopically labeled (SIL) peptide. A heavy peptide is a peptide that is bound to a non-radioactive, stable isotope, typically ¹³C (carbon-13), ¹⁵N (nitrogen-15), or ²H (deuterium), but not limited to. In general, heavy peptides have equivalent physiochemical properties and chemical reactivity to the light peptide being analyzed and behave differently from the light peptide due to the isotopic mass difference. These heavy peptides are used to quantify the absolute amount of the light peptide being analyzed. In the present invention, the “light peptide” refers to a peptide that is not labeled with an isotope, as opposed to a “heavy peptide,” and generally refers to the target peptide that is the subject of the quantitative analysis.

According to a specific implementation of the present invention, the preprocessing part converts the input training data into a heatmap having two channels, a light peptide channel and a heavy peptide channel for the target peptide to be quantified.

In the present invention, the term “heat map” refers to input data to a convolutional neural network that is processed and generated by a preprocessing unit. Typically, the input image data to a convolutional neural network consists of three channels of RGB, i.e., three two-dimensional array data, but the input data to the convolutional neural network of the present invention consists of two two-dimensional array data, each array containing transition chromatogram data of light and heavy peptides. A “heat map” is generated by processing or transforming the transition chromatogram data and a transition list extracted from the mass spectrometry data. In the present invention, the “transition list” refers to data containing information about which transitions are targeted in each target peptide subject to quantitation in the present invention, including information about the m/z values of the precursor ion and product ion pairs.

According to a specific implementation of the present invention, one axis of the heatmap is a Retention Time and the other axis is a Multiple Transition.

In the present invention, the term “retention time” refers to the time taken from the injection of a sample to its outflow in a gas or liquid chromatographic analysis, and the retention time can be used as an indicator for the identification of a substance because it generally has a unique value for each substance when conditions such as the type of column, column temperature, type of mobile phase and flow rate are constant.

According to a specific embodiment of the present invention, the preprocessing part performs data augmentation on the data for training, prior to processing the data for training.

In the present invention, the “data augmentation” refers to the process of increasing the diversity of a training set by applying random transformations, such as image rotation. This process amplifies the amount of data by utilizing a small amount of data, when the amount of data in the dataset is typically insufficient.

In the present invention, the “training set” refers to data used to train the algorithm of a training model, i.e., data used for training (i.e., repeatedly modifying weights), while “test set” refers to data used to evaluate the performance of the trained training model. In general, there are various augmentation methods for image data, such as cropping and random resizing, which involves cropping and resizing certain parts of the image, jittering, which involves randomly changing data such as color, rotating or flipping the image, and changing the brightness.

According to a specific implementation of the present invention, the data augmentation comprises at least one selected from the group consisting of Random Resizing, Cropping, Intensity Jittering, Retention Time Shifting, and Transition Rescaling.

In the present invention, the “random resizing” refers to a data enhancement method that reduces or increases the size of a transition chromatogram along the time axis to generate peak shapes at different scales.

In the present invention, the “cropping” refers to a data enhancement method that truncates the start and end of a transition chromatogram to alter the peak position on the time axis.

In the present invention, the “intensity jittering” refers to a method of data augmentation that intentionally adds noise.

In the present invention, the “retention time shifting” refers to a data augmentation method that modifies peak positions by adding blank signals before and after the transition chromatogram, as opposed to “cropping”.

In the present invention, the “Transition Rescaling” refers to a data augmentation method that adjusts the signals of a particular pair of transitions along the intensity axis.

According to a specific embodiment of the present invention, the training model comprises a Backbone Network and a plurality of Sub-networks, the Backbone Network is a Modified ResNet34, the Sub-networks include a sub-network for classifying Quantifiability of peak groups and a sub-network for performing Peak Boundary Regression.

In the present invention, the “Backbone Network” means one of the networks comprising a convolutional neural network that uses an image as input to extract a feature map that serves as the basis for the rest of the network.

In the present invention, the “Sub-networks” refers to networks that perform tasks such as object classification using the output of the Backbone Network as input.

In the present invention, the “ResNet” refers to a type of artificial neural network that solves the problem of gradient loss and gradient explosion, which occurs as the neural network grows deeper, using a “residual training” technique.

In the present invention, the “ResNet34” refers to a type of ResNet, which is a convolutional neural network that can be used for image classification and has 34 layers.

According to a specific implementation of the present invention, the Modified ResNet 34 comprises layers 0 to 5,
the layer 0 is a kernel size of 1×7, 32 channels, and a stride of 1×1;
the layer 1 is a kernel size of 3×7, 64 channels, and a stride of 1×1;
the layers 0 and 1 comprise two groups to be synthesized separately with the heavy peptide channel and the light peptide channel;
the layer 2 comprise a max pooling layer with a kernel size of 1×3 and a stride of 1×2, an adaptive mean pooling layer, and three residual blocks, each of which comprises two convolutional layers with a kernel size of 1×3 and 128 channels;
the layer 3 comprise three residual blocks, each of which comprises two convolutional layers with a kernel size of 1×3 and 128 channels;
the layer 4 comprise three residual blocks, each of which comprises two convolutional layers with a kernel size of 1×3 and 256 channels;
the layer 5 comprises three residual blocks, each of which comprises two convolutional layers with a kernel size of 1×3 and a channel count of 512.

In this specification, the term “Residual Block” refers to a unit block of a ResNet, which is structured in such a way that it adds inputs before passing parameters to the next layer.

In the present invention, the “channel” refers to the number of filters applied to the input data entering the convolutional layer. For example, a color image is three-dimensional data represented as three real-valued channels (RGB), which would have three channels.

In the present invention, the “kernel” refers to a set of weights, also referred to as a filter, and means a receptive field that performs the role of extracting a feature map that emphasizes regions of the image similar to the filter through a convolutional operation from the input data through a weight parameter (W) and passing it to the next layer. Accordingly, the term “kernel size” refers to the size of the kernel.

In the present invention, the “pooling layer” refers to a layer of a type commonly used in convolutional neural networks that downsamples each feature map computed by a convolutional operation. For example, maximum pooling, which is the most commonly used pooling size of 2, involves dividing the feature map into 2×2 dimensions and converting the maximum (or average in the case of average pooling) of the pixel values in each region into a new image with pixel values, thereby reducing the computation of the convolutional neural network.

In the present invention, the “stride” refers to a method that can reduce the computation of a convolutional neural network in addition to the pooling layer. Specifically, a stride is the interval at which the filter (kernel) moves over the input image. For example, a convolutional operation with a stride of 1 means that the filter performs the convolutional operation by moving one element of the input, while a stride of 2 means that the filter performs the convolutional operation by moving two elements of the input. Pooling has the disadvantage that the resolution of the image passed to the next layer is significantly reduced by half, resulting in a large loss of data, but stride reduces the amount of computation while ensuring that every element of the input affects the output, so unlike pooling, no information is lost.

According to a specific implementation of the present invention, the subnetwork has a kernel size of 1×3.

According to a specific implementation of the present invention, the post-processing part comprises comparing the similarity between the heavy peptide peak shapes and the light peptide peak shapes within the boundaries of the selected peaks to select a transition pair of the heavy peptide and the light peptide having the highest similarity.

According to a specific implementation of the present invention, the post-processing part further comprises selecting a transition pair of the light peptide by comparing the similarity of a mean profile of the heavy peptide peak shape and the light peptide peak shape, before comparing the similarity of the light peptide peak shape and the heavy peptide peak shape.

In the present invention, the “mean profile” refers to the average peak shape of the heavy peptide peaks. Since heavy peptides are always contained in a biological sample in fixed amounts, they are observed in the form of a uniform and clean signal without significant differences between samples, and the average peak shape of the corresponding heavy peptide peaks is derived and considered as a “representative peak shape”. By first comparing the peak shapes of the light peptides with the average profile of the corresponding heavy peptides, the light peptide peaks that are outliers can be preemptively removed, resulting in a more efficient selection of light peptide peaks with the highest similarity to the heavy peptides.

According to a specific implementation of the present invention, the similarity is calculated using dot-product similarity.

In the present invention, the “dot-product similarity” refers to a formula that can determine the degree of similarity of two vectors using the dot-product of the two vectors. Specifically, regardless of the magnitude of the two vectors, only the directional similarity can be determined, and the highest similarity is achieved when the two vectors have the same direction (θ=0) and the cos θ value is 1. The lowest similarity is achieved when the two vectors have opposite directions (θ=π) and the cos θ value is −1.

According to one aspect of the present invention, the present invention provides a method for selecting a peak for quantification of a target peptide from liquid chromatography mass spectrometry data which is not selected as peak for quantification, using the system of selecting peak of the present invention described above. The system may be, for example, a trained system of selecting peak.

In the present invention, the “trained” means that the training model has reached a desired level of predictive accuracy after iterative training with the input data (wherein one epoch is defined as one pass of the entire training data set through the neural network, and n epochs is defined as n iterations). Specifically, training is considered complete when there is no improvement over 30 epochs, but various numbers of epochs can be used as a benchmark, including but not limited to.

According to one aspect of the present invention, the present invention provides a computer program stored on a computer-readable recording medium, coupled with hardware, comprising executing the system of selecting peak to simultaneously select a plurality of peaks optimized for quantification of a plurality of target peptides.

The methods according to embodiments may be implemented in the form of program instructions that may be executed through various computer means and recorded on a computer-readable medium. The computer-readable medium may include program instructions, data files, data structures, and the like, singly or in combination. The program instructions recorded on the medium may be specifically designed and configured for the embodiment or may be known and available to those skilled in the computer software art. Examples of computer-readable recording media include magnetic media, such as hard disks, floppy disks, and magnetic tape; optical media, such as CD-ROMs and DVDs; magneto-optical media, such as floptical disks; and hardware devices specifically configured to store and execute program instructions, such as ROMs, RAMs, flash memory, and the like. Examples of program instructions include machine language code, such as that generated by a compiler, as well as high-level language code that can be executed by a computer using an interpreter or the like. The hardware device may be configured to operate as one or more software modules to perform the operations of the embodiments.

According to a specific implementation of the present invention, the computer program selects a peak for quantification of the target peptide selected by user input through a graphical user interface (GUI).

In the present invention, the “GUI” is also referred to as a “graphical user interface” or simply “graphical interface”, meaning a user interface that utilizes a mouse, icons, and windows.

### Advantageous Effects

The features and advantages of the present invention are summarized as follows:
(a) The present invention provides an automated peak selection system that is as accurate as human and expert peak selection in targeted proteomics, which previously required manual intervention by researchers, wasting a lot of time and resources.
(b) The training model of the present invention, or a computer program capable of executing it, may be useful for rapidly and accurately selecting optimized peaks for quantification of a plurality of target peptides entered as desired by a user via the program's GUI.

### BRIEF DESCRIPTION OF DRAWINGS

FIG. 1 illustrates the workflow of DeepMRM, a training model of the present invention for detecting peaks of target peptides. Given a transition list and MRM/PRM data as input, the transition chromatograms of heavy and light peptides are provided as two-channel heatmap images, and the multi-scale 1D feature map extracted by the Backbone Network is processed by two sub-networks. The two subnetworks are a Classifier that determines whether a candidate peak is quantified or not, and a Regressor that detects the boundaries of the candidate peaks.

FIG. 2 illustrates the graphical user interface (GUI) of the DeepMRM desktop software.

FIG. 3 is a bar graph showing the average precision (AP) and average recall (AR) scores for the benchmark dataset.

FIG. 4 is a scatter plot comparing the results of the heavy/light ratio calculated by the manually annotated peaks and the peaks detected by DeepMRM.

FIGS. 5A, 5B, 5C, 5D and 5E provide an illustration of the Data Augmentation method. FIG. 5A is the original transition chromatogram. FIG. 5B is a transition chromatogram with Random Resizing and Cropping applied. FIG. 5C is a transition chromatogram with random Retention Time Shifting applied. FIG. 5D is a transition chromatogram with Transition Rescaling applied. FIG. 5E is a transition chromatogram with Intensity Jittering applied. Manual peak boundaries are shown as dashed lines.

FIGS. 6A, 6B, 6C, 6D, 6E compare the quantification performance of the Skyline software with DeepMRM with and without the mProphet algorithm as a quality control method applied to the results. FIGS. 6A, 6B and 6C show the relative quantification and distribution of heavy peptide abundances in the noisy dataset, and FIGS. 6D, 6E and 6F show the relative quantification and distribution of heavy peptide abundances in the complex background dataset. In experiments on the complex background dataset, mProphet does not filter any results, so there is no difference between Skyline Default and Skyline FDR 5%. The red dashed line represents the abundance of heavy peptides, and the centerline, edges, and whiskers in the boxplot represent the median, 1st and 3rd quartiles, and 1.5× interquartile range, respectively. Outlier points outside the whiskers are indicated by a dot symbol.

FIGS. 7A, 7B, 7C, 7D, 7E and 7F show the distribution of absolute percentage error for two dilution series datasets: FIGS. 7A, 7B and 7C show the distribution of absolute percentage error for the noisy dataset, and FIGS. 7D, 7E and 7F show the distribution of absolute percentage error for the complex background dataset. In the experiments on the complex background dataset, mProphet does not filter any results, so there is no difference between Skyline Default and Skyline FDR 5%. In the boxplot, the centerline, edges, and whiskers represent the median, 1st and 3rd quartiles, and 1.5× interquartile range, respectively. Outlier points outside the whiskers are marked with a dot symbol.

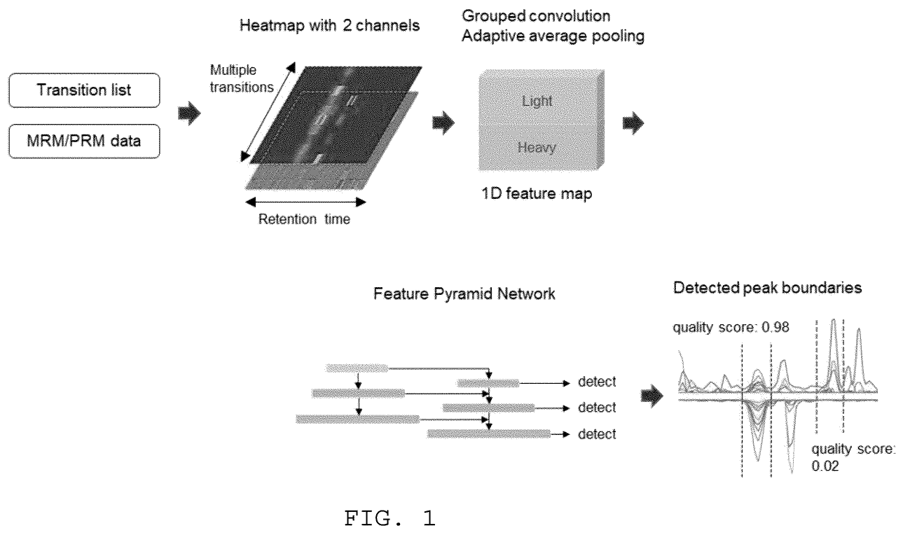

### MODE FOR INVENTION

Hereinafter, the present invention will be described in more detail by way of examples These examples are only for illustrating the present invention in more detail, and it will be apparent to those skilled in the art that the scope of the present invention according to the subject matter of the present invention is not limited by these examples.

### Example

### Experimental and Analytical Methods

### Datasets

### Collecting Datasets

Four LC-MRM/PRM/DIA-MS experimental datasets were obtained for training and evaluation. One dataset was generated using in-house samples, while the other three datasets were downloaded from public repositories. Only the internal dataset was used for training and the external dataset was used for evaluation (Table 1).

### Pancreatic Ductal Adenocarcinoma Dataset (PDAC-MRM)

Tissue samples from 66 pancreatic ductal adenocarcinoma patients were cryopulverized, lysed, and trypsin-digested by a modified Filter-Associated Sample Preparation (FASP) method (see reference 1). Pierce BCA assay kits (Thermo Scientific) were used according to the manufacturer's instructions for protein and peptide quantification. For the Stable Isotope Labeled (SIL) peptides, each of the 153 peptides was a stable isotope labeled with a ¹³C₆ lysine or ¹³C₆ arginine analogue at C-terminal lysine or arginine with a mass difference of 6.02013 Da compared to the mass of the corresponding endogenous peptide sequence, and the purity of the SIL peptides was greater than 95%. The purified SIL peptide was analyzed by the AAA-MS method for absolute quantification of amino acids (see reference 2). MRM conditions were optimized for peptide amount, collision energy for CS+2 and CS+3 precursor ions, and retention time for 153 target peptides. If a peptide had similar intensities for CS+2 and CS+3 precursor ions, both transitions were included in the target list. However, only one precursor with higher intensity and less interference was subsequently selected for quantification. LC-MRM-MS experiments were performed using an in-house built dual online nano-flow LC system (Ultimate 3000 NCP-3200RS, Thermo Fisher Scientific) coupled to an Agilent 6495C triple quadrupole mass spectrometer platform (see reference 3). The injection amount of PDAC tissue peptides was 5 μg. Dynamic MRM was performed on the three best y-ion transitions of 153 targets, excluding y1 and y2 ions, with a time window of 3-5 min. A spray voltage of 2500 V, a dry gas flow of 5 L min⁻¹ and a gas temperature of 225° C. were used. Both Q1 and Q3 resolutions were set to unity; the collision energy was set to 10-40 based on previously optimized values; and a cycle time of 800 ms was used. Q1 and Q3 resolutions were both set to Unit, collision energy was set to 10-40 based on previously optimized values, and a Cycle Time of 800 ms was used. The column was prepared in-house using Jupiter C18, 3 μm, 300 Å particles, 75 μm×50 cm (Phenomenex), and the column temperature was maintained at 60° C. A 60-min gradient (10%-40% solvent B over 47 min; 40%-80% over 5 min; 80% for 6 min and 10% for 2 min; 400 nL min⁻¹) was used for each experiment. Solvent A was 0.1% formic acid in water and solvent B was 0.1% FA in acetonitrile (ACN). The 198 LC-MRM-MS data generated were analyzed with Skyline version 21.1.0.1464 and transitions were manually inspected using the following evaluation criteria: equal retention time between heavy and light peptides, peak shape, intensity ratio consistency across transitions, and elimination of transitions with peak interference. For quantifiable peptides, the ratio of heavy/light peptides was determined based on peak area. A total of 30,294 transition group records were annotated, of which 19,230 records could be quantified.

### External Datasets (EOC-MRM and P100-PRM)

Three external datasets were obtained from the MassIVE repository. The first dataset used MRM data of biomarkers for epithelial ovarian cancer (EOC-MRM) samples (see reference 5; MassIVE identifier: MSV000084048). The second dataset used a PRM validation dataset of ˜100 phosphopeptide (P100-PRM) samples generated for the Library of Integrated Network-based Cellular Signatures (LINCS) project (see reference 6; MassIVE identifier: MSV000079524). As a third dataset, we used DIA data from the same phosphoproteomics sample (P100-DIA) as the dataset used to evaluate the accuracy of expert manual curation of the Avan-Garde (AvG) tool (see reference 7; MassIVE identifier: MSV000085540). In benchmark testing of the present invention, the inventors evaluated the present invention (DeepMRM) with the AvG open curation dataset. For all these datasets, Skyline files were generated during the quantification analysis. For the P100-PRM dataset, the quantification results were filtered and normalized by our in-house downstream analysis protocol (see reference 6). Since filtered quantification results were not available, Skyline's “dotp” score of 0.7 was used to filter out unreliable measurements. This filtering resulted in the exclusion of 2,117 of the 13,629 peak groups. Through manual inspection of the excluded peak groups, we found that the majority of the excluded peaks did not have heavy or light peptide signals.

TABLE 1
MRM/PRM datasets for training and benchmark testing.

| | | | #Run | #target | #Transition | #annotated |
|---|---|---|---|---|---|---|
| Datasets | Instruments | Sample | LC/MS | Peptides | per peptide | peakgroup |
| PDAC- | 6495C triple quadruple | Pancreatic | 198 | 153 | 3 | 30,294 |
| MRM | (Agilent) | Ductal | | | | |
| | | Adenocarcinoma | | | | |
| | | tissue | | | | |
| EOC- | 4000QTRAP and | Blood plasma | 463 | 78 | 2-5 | 20,729 |
| MRM | 5500QTRAP (Sciex) | | | | | |
| | TSQVantage | | | | | |
| | (ThermoFisherScientific) | | | | | |
| P100- | Q-Exactive | MCF7, PC3, | 144 | 95 | 3-17 | 13,629 |
| PRM | (Thermo Fisher | and HL60 | | | | |
| | Scientific) | | | | | |
| P100- | Q-Exactive HF Plus | MCF7, PC3, | 96 | 95 | 3-25 | 9,025 |
| PRM | (ThermoFisherScientific) | and HL60 | | | | |
| Dilution | TSQ Quantum | Kc167 | 275 | 43 | 4-9 | N/A |
| series | Ultra EMR | | | | | |
| | (ThermoFisherScientific) | | | | | |

The PDAC-MRM dataset was split into training, validation, and test sets in a ratio of 8:1:1, while the three external datasets, EOC-MRM, P100-PRM, and P100-DIA, were used for evaluation purposes only.

### Peak Detection Model

### Data Preprocessing

Given input data including LC-MS data and a Target List, DeepMRM, the peak detection model of the present invention first extracted Transition Chromatograms for the target precursor ions and then converted them into a two-channel heatmap image comprising a channel for light peptides and a channel for heavy peptides. The entire transition chromatogram acquired for the target peptide was extracted unless a Reference Retention Time was specified along with a Time Window for the target peptide. Linear Interpolation was applied to all transition chromatograms so that they all had the same length and the same scan interval of 0.7 seconds. The size of the heatmap image corresponds to [2, number of transitions, chromatogram length]. As a final preprocessing step, each Transition Pair was scaled to the range 0-1.

### Model Architecture

The structure of the peak detection model is based on RetinaNet, a neural network model for object detection (see reference 8). The network consists of a Backbone Network for feature extraction and two smaller sub-networks. The first sub-network classifies the quantifiability of peak groups in the extracted features, and the second sub-network performs Peak Boundary Regression. The inventors chose ResNet34 as the Backbone Network and modified it to be suitable for the peak selection problem (Table 2).

TABLE 2
Structure of the backbone network of the inventive training model
DeepMRM, a variant of ResNet34

| Layer names | Convolution Layers | Feature Map |
|---|---|---|
| conv0 | 1x7, 32, stride 1x1, 2 groups | |
| conv1 | 3x7 64, stride 1x2, 2 groups | |
| conv2 | 1x3, max pulling, stride 1x2 | C1 |
| | Adaptive average pooling | |
| | $[ \begin{matrix}1 \times 3 , 64 \\ 1 \times 3 , 64\end{matrix} ] \times 3$ | |
| conv3 | $[ \begin{matrix}1 \times 3 , 128 \\ 1 \times 3 , 128\end{matrix} ] \times 3$ | C2 |
| conv4 | $[ \begin{matrix}1 \times 3 , 256 \\ 1 \times 3 , 256\end{matrix} ] \times 3$ | C3 |
| conv5 | $[ \begin{matrix}1 \times 3 , 512 \\ 1 \times 3 , 512\end{matrix} ] \times 3$ | C4 |

First, a new convolutional layer (conv0) with a kernel size of 1×7 was placed on top of the first convolutional layer (conv1) in the backbone. Batch Normalization followed by the ReLU function was applied to conv0. We changed the Kernel Size of Conv1 from 7×7 to 3×7 and adjusted the Stride and Padding Sizes accordingly. For the first two convolutional layers, we applied Grouped Convolutions so that the heavy and light peptide channels were convolved separately. Before the first Residual Block, an Adaptive Average Pooling Layer was inserted so that the height of the Feature Map is 1. In subsequent Residual Blocks, the kernel and padding sizes were adjusted to 1×3 and 0×1, respectively. This ensured that all feature maps output from the backbone were generated as one-dimensional vectors. The two subnetworks also used convolutional layers with a kernel size of 1×3 and padding size of 0×1. For the Regressor, the final output channel size is changed to twice the number of anchors since it only needs to predict 2 points for the peak boundary instead of 4 points in the box for the Bounding Box. Anchors were created with a Single Scale for each Feature Level. The Classifier Subnetwork was set up with three classification problems: Background, Poor Peak, and Quantifiable Peak. In the case of poor peaks, the peak boundaries were often not clear. Therefore, we halved the Regression Loss for poor peak boundaries.

### Post-Processing Model Output: Transition Selection

Since some transition chromatograms within the detected boundaries are often affected by interference or noise, the present invention (DeepMRM) performed post-processing on the model output to remove outlier transitions from the quantification process in order to find the optimal transition pairs for accurate quantification. For the detected peak boundaries, the Mean Profile of the Heavy Peak shape was first calculated. Then, the Dot-product Similarity between the Light Peak shape and the Mean Profile was calculated. Selected Light Transition values were compared to the corresponding Heavy Transition Pairs. Finally, the Heavy Transition Pairs and Light Transition Pairs with the highest Dot-product Similarity were selected for quantification

### Data Augmentation

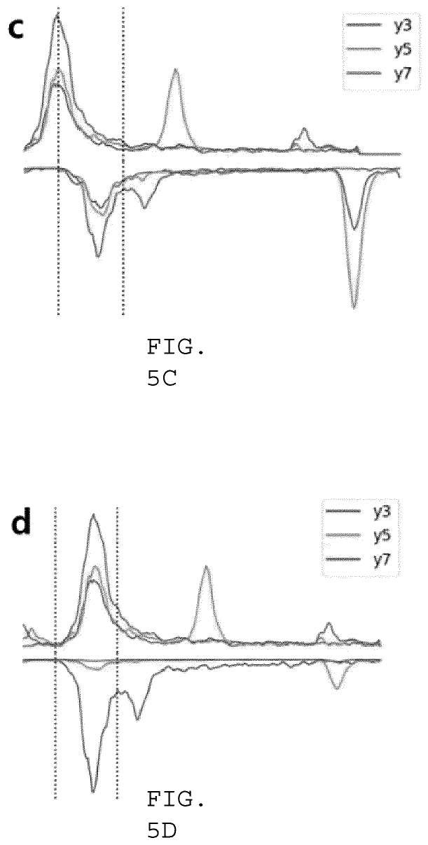

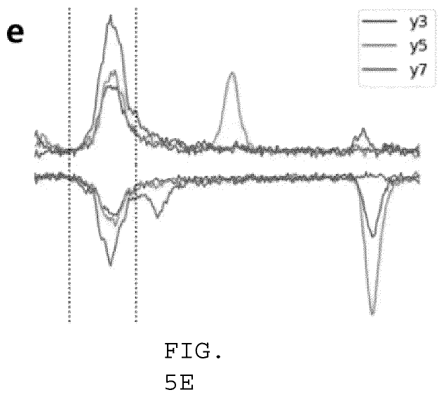

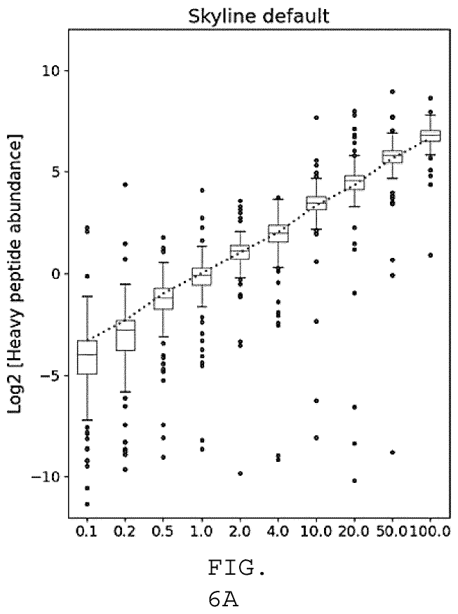

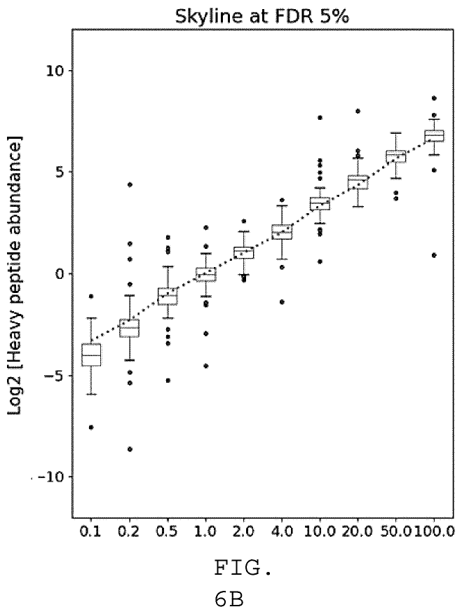

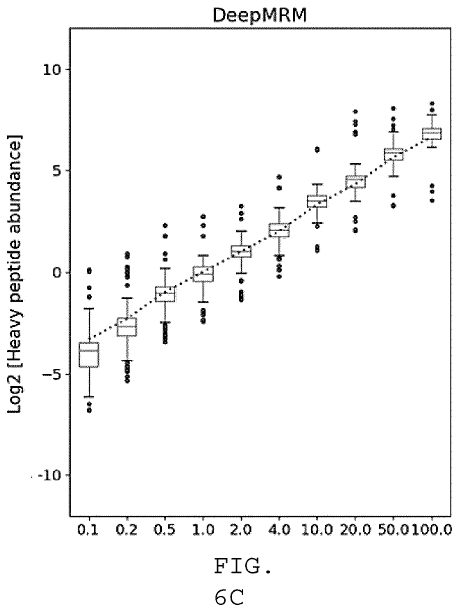

To improve the robustness and applicability of the present invention (DeepMRM), a data augmentation strategy is adopted for model training. Data augmentation methods include Random Resizing, Cropping, Intensity Jittering, Retention Time Shifting, and Transition Rescaling (FIGS. 5a to 5e). Retention Time Shifting was used to break the alignment of the light transition peak and heavy transition peak, and Transition Rescaling was used to make the light/heavy ratio inconsistent across transitions. During the data augmentation process, Label Data was also transformed accordingly. Also, to make the model invariant to the order of input transitions, they were randomly shuffled during training (e.g., (y3, y5, y9)→(y9, y3, y5)). All of the above augmentation methods were applied before the heatmap images were generated.

### Training

The in-house dataset (PDAC-MRM) was split into training; validation; and test sets in a ratio of 8:1:1, respectively. The model of the present invention was trained using the Adam optimizer with default parameters (lr=1.e-3, β1=0.9, β2=0.999). Training was performed for 100 epochs with a Batch Size of 512 using an NVIDIA Geforce 3090 GPU. The training rate was decreased by 0.5 every 10 epochs. Training was stopped when there was no improvement for more than 30 epochs.

Quantitative Efficiency Comparison with Skyline Software

### How to Compare Quantitative Efficiency

First, the inventors evaluated the quantification performance of the inventive peak selection system (DeepMRM) using two series dilution datasets from a public MRM dataset (Nasso, S., Goetze, S., and Martens, L., 2015) and compared it to the quantification performance of Skyline software. In this dataset, the abundances of heavy peptides varied from 0.1 to 100 femtomoles, while the abundances of light peptides remained constant. Absolute quantification was achieved by an external calibration curve method. The inventors used Pearson's correlation coefficient (PCC) and Spearman's rank correlation coefficient (SPC) to assess the linear relationship between the known and measured abundance of heavy peptides, and the absolute percentage error was also calculated. The quantification results of the Skyline software, reported in previous benchmark tests, were analyzed through two scenarios: the original results (Skyline's default) and the results filtered by the mProphet scoring model (Skyline with an FDR of 5%) based on decoy transitions. For this analysis, each technical replicate was considered an independent sample, i.e., quantification values for the same peptide in a replicate were considered separate observations.

### Dilution Series Dataset

The MRM dataset generated to benchmark the quantification algorithm was downloaded from the PeptideAtlas repository (dataset identifier: PASS00456). The dataset was generated from two dilution series using different sample and collection conditions with 43 SIL peptides added: a complex background to obtain a complex background dataset, and a noisy dataset under suboptimal conditions with no background. In these datasets, the abundance of heavy peptides varied from 0.1 to 100 femtomoles, while the abundance of light peptides was fixed. The quantification results reported in the benchmark study are also available for download at https://github.com/saranasso/Ariadne.

### Experimental Results

### Evaluation

For evaluation, four data sets from LC-MRM/PRM/DIA-MS experiments were used as described above in the Data Acquisition Table of Contents. When extracting Transition Chromatograms from the P100-DIA data set, a retention time window of 20 minutes was used with a reference retention time specified in the spectral library. For other MRM and PRM data sets, the entire transition chromatogram acquired for the target peptide was extracted and fed into DeepMRM. Before extracting the chromatograms, all PRM and DIA spectra were centroided and the width of the extraction window was set to 20 ppm. We calculated Average Recall (AR) and Average Precision (AP), which are conventional metrics used in the object detection task (see reference 9). Only quantifiable peaks were considered in the evaluation, as poor peaks were mostly indistinguishable from the background. A detected peak group was considered a true positive only if the Intersection over Union (IoU) between it and the manually annotated peak group (i.e. ground-truth) was greater than a certain threshold. Chromatogram peaks often have long tails, which can lead to large deviations in determining the end of the peak, but since these deviations do not significantly affect the peak area, we used a less stringent IoU threshold of 0.3 rather than the more common IoU threshold of 0.5 in object detection studies. AR was calculated for the top 1 and top 3 candidates per heatmap image (AR1 and AR3). To validate the quality of the true-positive peaks, we also evaluated the Pearson correlation coefficient (PCC) and Spearman's correlation coefficient (SPC) between the Light/Heavy Ratio obtained by manual annotation and the value obtained by DeepMRM.

### Result

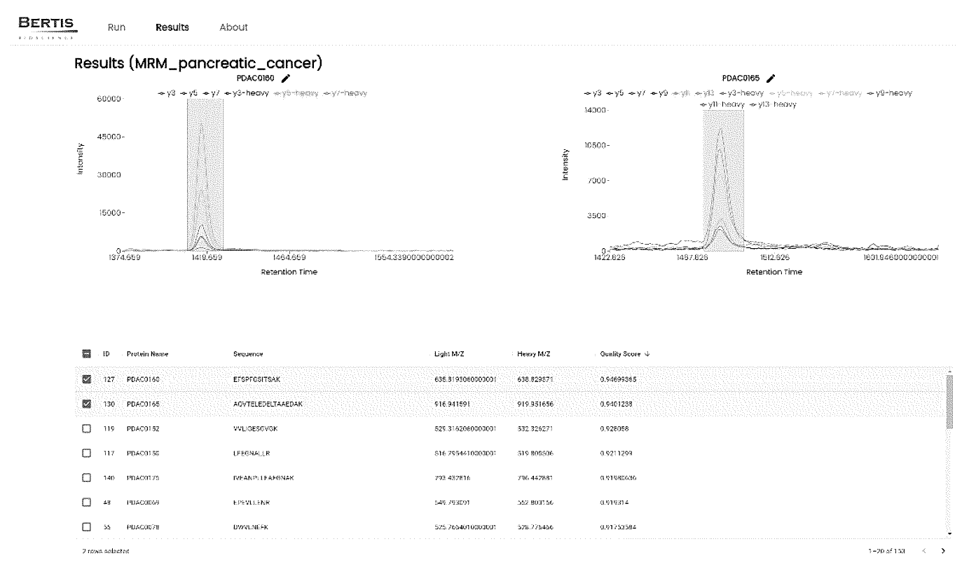

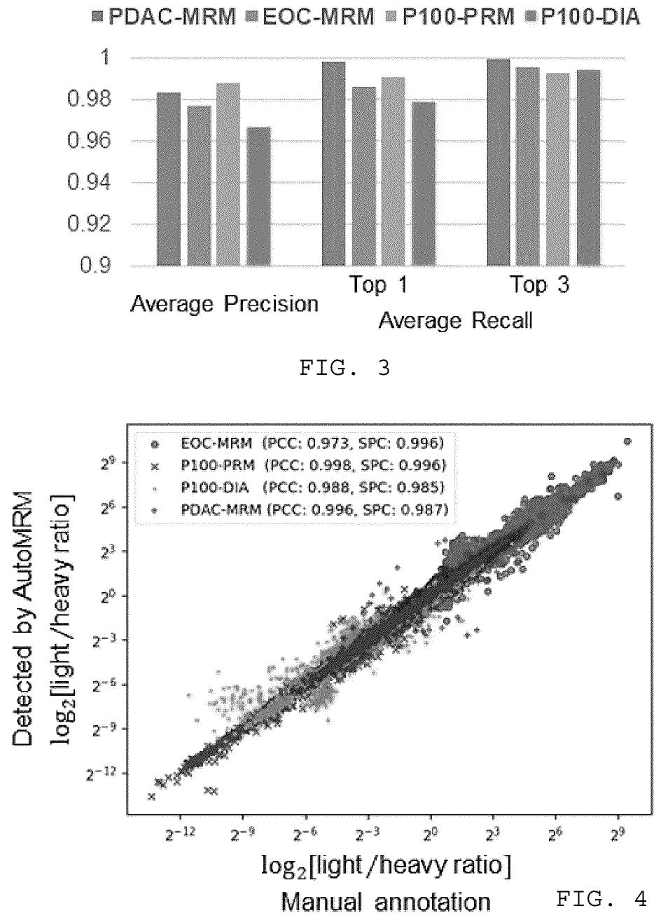

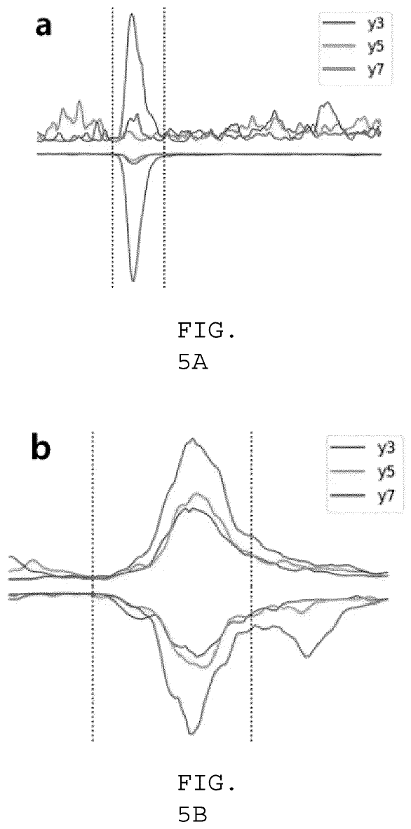

The inventors benchmarked DeepMRM using the aforementioned dataset (PDAC-MRM) and three external datasets, an MRM dataset used in epithelial ovarian cancer research (EOC-MRM; see reference 8), and PRM and DIA datasets used to profile Phosphosignaling Responses (P100-PRM and P100-DIA; see references 9 and 10) (Table 2). On these datasets, we found that the present invention (DeepMRM) demonstrated an average precision (AP) of 96-99% and an average recall (AR) of 98-100% (FIG. 3). We also found that the Modified Architecture and Data Augmentation methods improved the model's Accuracy and Robustness (AP and AR increased by up to 1% and 0.6%, respectively, Table 3). When comparing the light/heavy ratios between manually annotated peaks and those detected by the present invention (DeepMRM), the Pearson's correlation coefficient (PCC) and Spearman's rank correlation coefficient (SPC) were 0.97-1.0 and 0.98-1.0, respectively (FIG. 4). Considering the expected deviations in manual labeling, the accuracy of the present invention (DeepMRM) was found to be similar to that of human experts. Furthermore, the time and resources required for data interpretation were significantly reduced when using the present invention (DeepMRM). Manual inspection of the 66 samples in the PDAC-MRM dataset took more than 600 hours, while the same task was completed in 232 seconds on a desktop computer (AMD Ryzen 7 5800X, 3.8 GHZ, 32 GB RAM) using the present invention (DeepMRM). Furthermore, human experts were able to verify and adjust DeepMRM's peak selection very efficiently using the GUI (FIG. 2). In summary, the present invention (DeepMRM) is a robust and highly accurate peak detection model for interpreting Targeted Proteomics Data, assisted by a User Interface that visualizes the detection results. In addition to standalone software, DeepMRM is also available as an integration into Skyline software.

TABLE 3
Comparison of training model (DeepMRM) performance with and
without modified Backbone Network and data augmentation.

| Modified | Data | PDAC-MRM | | | EOC-MRM | | | P100-PRM | | | P100-DIA | | |
|---|---|---|---|---|---|---|---|---|---|---|---|---|---|
| backbone | enrichment | AP30 | AR1 | AR3 | AP30 | AR1 | AR3 | AP30 | AR1 | AR3 | AP30 | AR1 | AR3 |
| | | 0.983 | 0.995 | 0.995 | 0.977 | 0.981 | 0.989 | 0.967 | 0.976 | 0.980 | 0.934 | 0.945 | 0.984 |
| | ◯ | 0.984 | 0.998 | 0.998 | 0.977 | 0.989 | 0.997 | 0.976 | 0.988 | 0.991 | 0.857 | 0.896 | 0.980 |
| ◯ | | 0.986 | 0.996 | 0.997 | 0.977 | 0.981 | 0.991 | 0.976 | 0.985 | 0.987 | 0.934 | 0.944 | 0.981 |
| ◯ | ◯ | 0.984 | 0.998 | 0.998 | 0.986 | 0.991 | 0.997 | 0.977 | 0.982 | 0.988 | 0.942 | 0.956 | 0.985 |

### Windows Desktop Applications

DeepMRM is packaged as a Windows desktop application that helps users run peak detection jobs and visualize results. The graphical user interface (GUI) of the desktop application allows users to quickly test detection results alongside input transition chromatograms, and multiple samples can be loaded together to easily compare all results for a target peptide. The desktop application supports the community standard for mass spectrometry data (mzML10).

Quantified Performance Comparison with Skyline Software

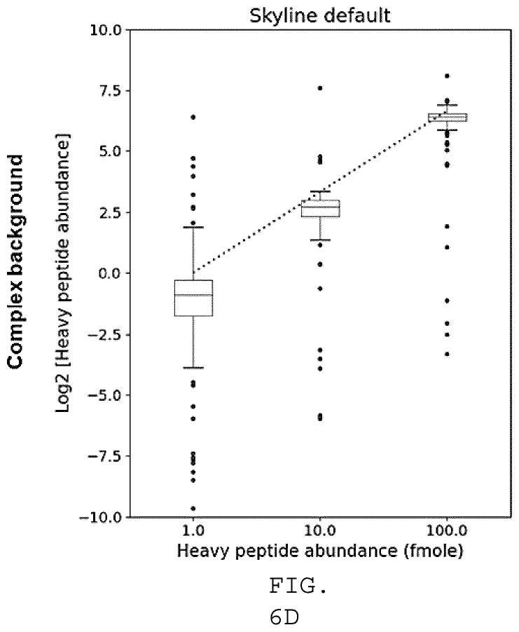

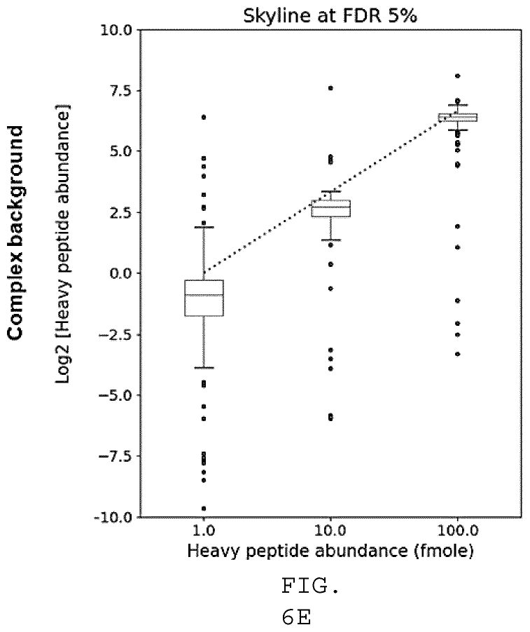

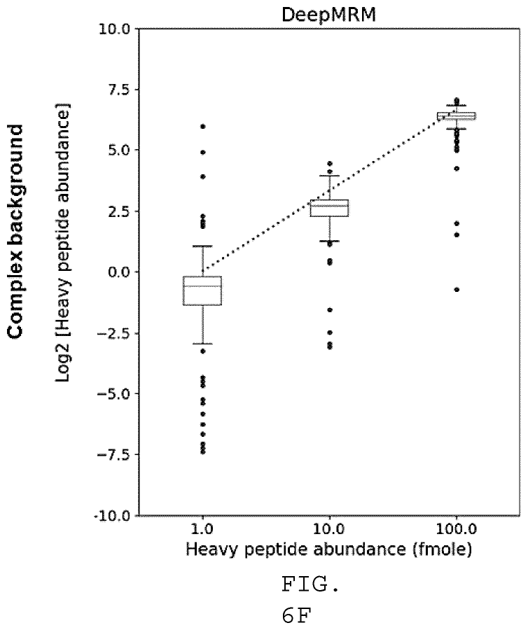

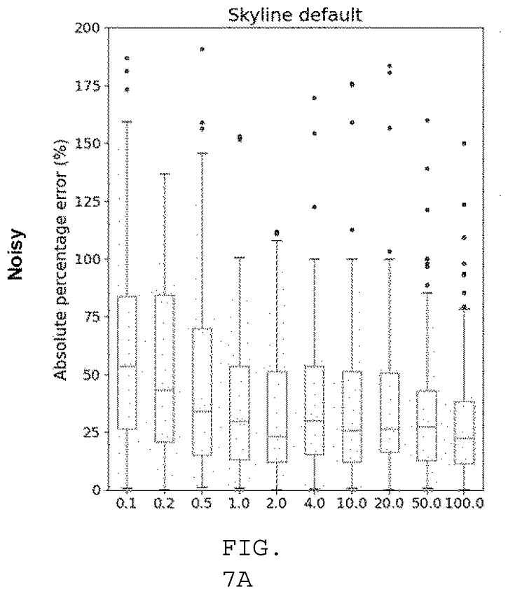

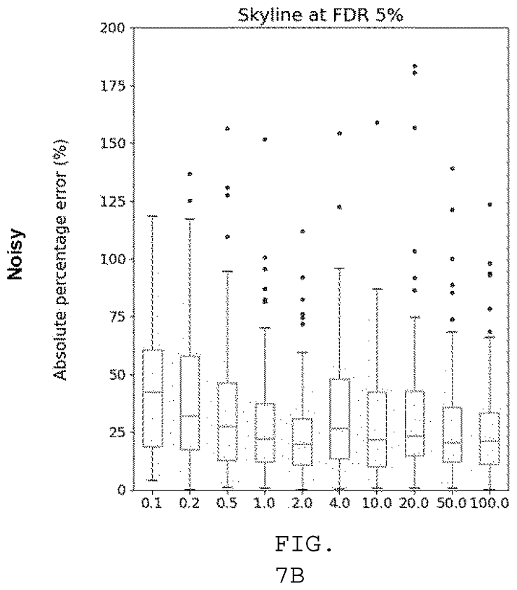

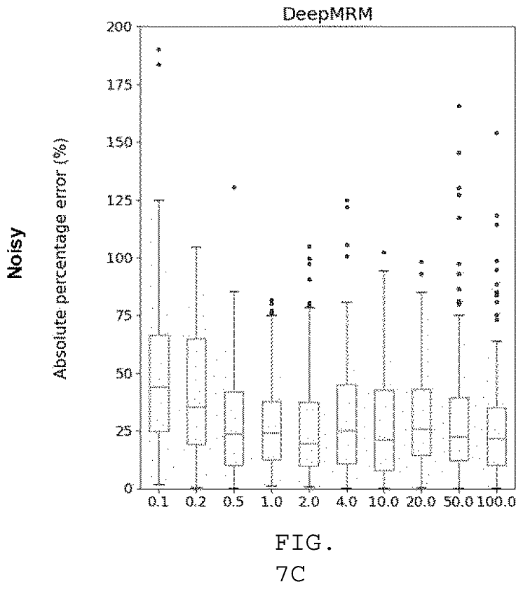

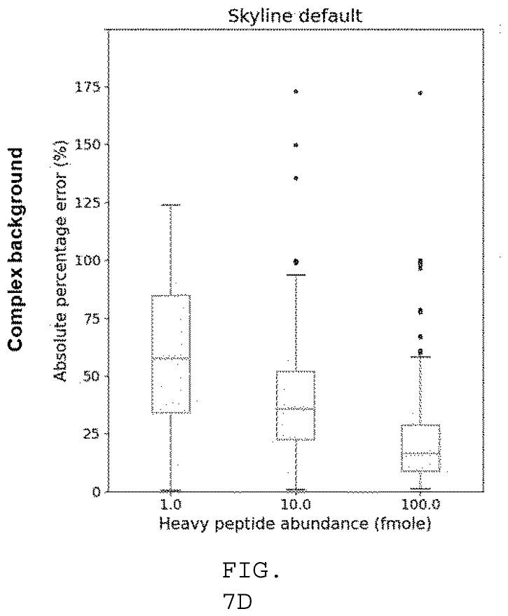

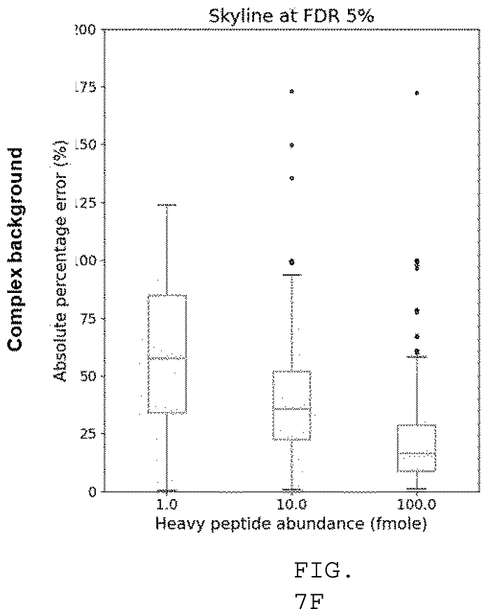

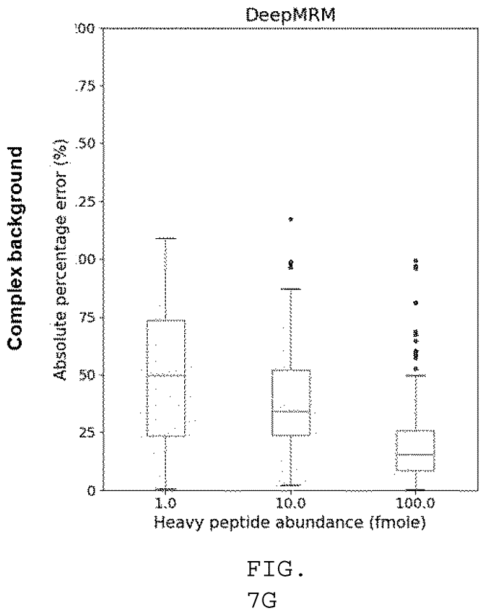

DeepMRM detected a larger number of quantifiable peak groups compared to Skyline software (hereafter, Skyline Default) and; Skyline software filtered with a false discovery rate of 5% using the mProphet algorithm based on decoy transitions (hereafter, Skyline FDR 5%) (FIG. 6). Furthermore, when evaluating the correlation and absolute error between the known and measured abundance of target peptides, DeepMRM showed higher average correlation coefficients (Table 4 and FIG. 6) and lower mean absolute percentage error (MAPE) than Skyline Default and Skyline FDR 5% (FIG. 7).

TABLE 4
Quantification performance of DeepMRM and Skyline compared
by average correlation coefficient and absolute percentage
error for 43 peptides in the target.

| | | Peak Group | PCC | SPC | MAPE |
|---|---|---|---|---|---|
| Noise | Skyline Default | 1287 | 0.9284 | 0.9413 | 68.09 |
| | Skyline FDR 5 | 971 | 0.9482 | 0.9723 | 50.99 |
| | DeepMRM | 1266 | 0.9760 | 0.9873 | 46.59 |
| Complex | Skyline Default | 386 | 0.9683 | 0.9237 | 94.95 |
| Backgrounds | Skyline FDR 5 | 386 | 0.9683 | 0.9237 | 94.95 |
| | DeepMRM | 372 | 0.9842 | 0.9287 | 68.09 |

While the foregoing has been described in detail with reference to specific features, it will be apparent those skilled in the art that these descriptions represent only preferred embodiments and are not intended to limit the scope of the invention. Accordingly, the substantial scope of the invention is defined by the appended claims and their equivalents.

### REFERENCES

1. Wiśniewski, J. R., Zougman, A., Nagaraj, N. & Mann, M. Universal sample preparation method for proteome analysis. Nature Methods 2009 6:5 6, 359-362 (2009).
2. Louwagie, M. et al. Introducing AAA-MS, a rapid and sensitive method for amino acid analysis using isotope dilution and high-resolution mass spectrometry. Journal of Proteome Research 11, 3929-3936 (2012).
3. Lee, H. et al. A simple dual online ultra-high pressure liquid chromatography system (sDO-UHPLC) for high throughput proteome analysis. Analyst 140, 5700-5706 (2015).
4. MacLean, B. et al. Skyline: an open source document editor for creating and analyzing targeted proteomics experiments. Bioinformatics 26, 966-968 (2010).
5. Hüttenhain, R. et al. A Targeted Mass Spectrometry Strategy for Developing Proteomic Biomarkers: A Case Study of Epithelial Ovarian Cancer. Molecular & Cellular Proteomics: MCP 18, 1836 (2019).
6. Abelin, J. G. et al. Reduced-representation phosphosignatures measured by quantitative targeted MS capture cellular states and enable large-scale comparison of drug-induced phenotypes. Molecular and Cellular Proteomics 15, 1622-1641 (2016).
7. Vaca Jacome, A. S. et al. Avant-garde: an automated data-driven DIA data curation tool. Nature Methods 2020 17:12 17, 1237-1244 (2020).
8. Lin, T. Y., Goyal, P., Girshick, R., He, K. & Dollar, P. Focal Loss for Dense Object Detection. IEEE Transactions on Pattern Analysis and Machine Intelligence 42, 318-327 (2017).
9. Lin, T.-Y. et al. Microsoft COCO: Common Objects in Context. Proceedings of the IEEE Computer Society Conference on Computer Vision and Pattern Recognition 3686-3693 (2015).
10. Martens, L. et al. mzML—a community standard for mass spectrometry data. Mol Cell Proteomics 10, R110.000133 (2011).

1. A system for selecting a peak for the quantification of target peptides in liquid chromatography mass spectrometry (LC-MS), comprising:
a preprocessing part that processes the input data for training;
a training part comprising a convolutional neural network (CNN) training model that learns to detect a boundary of a peak optimized for quantification of a target peptide using training data processed in the preprocessing part as input;
a post-processing part that processes the output of the training part; and
a determining part for selecting a peak for quantification of a target peptide using the output of the above post-processing part.

2. The system of claim 1, wherein the mass spectrometry is performed by a method selected from the group consisting of Multiple Reaction Monitoring (MRM), Parallel Reaction Monitoring (PRM), Data-Dependent Acquisition (DDA), and Data-Independent Acquisition (DIA).

3. The system of claim 1, wherein the data for training is a result of liquid chromatography mass spectrometry with the predetermined peaks for quantification.

4. The system of claim 3, wherein the results of the mass spectrometry comprise a transition value of the light peptide and a transition value of the heavy peptide for the target peptide to be quantified.

5. The system of claim 1, wherein the preprocessing part converts the input training data into a heatmap having two channels, a light peptide channel and a heavy peptide channel for the target peptide to be quantified.

6. The system of claim 5, wherein one axis of the heatmap is a Retention Time and the other axis is a Multiple Transition.

7. The system of claim 1, wherein the preprocessing part performs data augmentation on the data for training, prior to processing the data for training.

8. The system of claim 7, wherein the data augmentation comprises at least one selected from the group consisting of Random Resizing; Cropping; Intensity Jittering; Retention Time Shifting; and Transition Rescaling.

9. The system of claim 1, wherein the training model comprises a Backbone Network and a plurality of Sub-networks, the Backbone Network is a Modified ResNet34, the sub-networks include a sub-network for classifying Quantifiability of a group of peaks and a sub-network for performing Peak Boundary Regression.

10. The system of claim 9, wherein the modified ResNet34 comprises layers 0 to 5,
the layer 0 is a kernel size of 1×7, 32 channels, and a stride of 1×1;
the layer 1 is a kernel size of 3×7, 64 channels, and a stride of 1×1;
the layers 0 and 1 comprise two groups to be synthesized separately with the heavy peptide channel and the light peptide channel;
the layer 2 comprise a max pooling layer with a kernel size of 1×3 and a stride of 1×2, an adaptive mean pooling layer, and three residual blocks, each of which comprises two convolutional layers with a kernel size of 1×3 and 128 channels;
the layer 3 comprise three residual blocks, each of which comprises two convolutional layers with a kernel size of 1×3 and 128 channels;
the layer 4 comprise three residual blocks, each of which comprises two convolutional layers with a kernel size of 1×3 and 256 channels;
the layer 5 comprises three residual blocks, each of which comprises two convolutional layers with a kernel size of 1×3 and a channel count of 512.

11. The system of claim 9, wherein the sub-network has a kernel size of 1×3.

12. The system of claim 1, wherein the post-processing part comprises comparing the similarity between the heavy peptide peak shapes and the light peptide peak shapes within the boundaries of the selected peaks to select a transition pair of the heavy peptide and the light peptide having the highest similarity.

13. The system of claim 12, wherein the post-processing part further comprises selecting a transition pair of the light peptide by comparing the similarity of a mean profile of the heavy peptide peak shape and the light peptide peak shape, before comparing the similarity of the light peptide peak shape and the heavy peptide peak shape.

14. The system of claim 12, wherein the similarity is calculated using a dot-product similarity.

15. A method for selecting a peak for quantification of a target peptide from liquid chromatography mass spectrometry data which is not selected as peak for quantification, using the system of selecting peak of claim 1.

16. A computer program stored on a computer-readable recording medium, coupled with hardware, comprising executing the system of claim 1 to simultaneously select a plurality of peaks optimized for quantification of a plurality of target peptides.

17. The computer program of claim 16, comprising selecting a peak for quantification of the target peptide for a target peptide selected by user input through a graphical user interface (GUI).
